SEQUENCE LISTING (4 sequences)

SEQ ID NO: 1 - DNA, 24 bp, Artificial Sequence
  Description of Artificial SequencePCR primer entB3′-5′
gggctgcagacatttttaccgctg

SEQ ID NO: 2 - DNA, 26 bp, Artificial Sequence
  Description of Artificial SequencePCR primer entB5′-3′
tatggatccacgcgcatcagcctgaa

SEQ ID NO: 3 - DNA, 23 bp, Artificial Sequence
  Description of Artificial SequencePCR primer entC3′-5′
tgcggatcctcgctccttattgc

SEQ ID NO: 4 - DNA, 24 bp, Artificial Sequence
  Description of Artificial SequencePCR primer entC5′-3′
ggcgagctcattattaaagccttt

(19) United States
(12) Patent Application Publication
(10) Pub. No.: US 2003/0224495 A1
(43) Pub. Date: Dec. 4, 2003

(54) Method for the improved production and isolation of trans-dihydroxycyclohexadiene carboxylic acids and/or derivatives thereof and genetically modified organism suitable therefor

(76) Inventors: Dirk Franke, Seiwerath (DE); Georg Sprenger, Julich (DE); Ralf Takors, Merzenich (DE); Michael Muller, Julich (DE); Markus Halfar, Julich (DE); Rolg Muller, Wolfenbuttel (DE); Jorg Thommes, Julich (DE); Volke Lorbach, Duren (DE)

Correspondence Address: KLAUS J. BACH & ASSOCIATES, 4407 TWIN OAKS DRIVE, MURRYSVILLE, PA, 15668, US

(21) Appl. No.: 10/377,169

(22) Filed: Feb. 28, 2003

Related U.S. Application Data
(63) Continuation-in-part of application No. PCT/EP01/09980, filed on Aug. 30, 2001.

(30) Foreign Application Priority Data
Aug. 30, 2000 (DE) 100 42 535.6

(51) Int. Cl. C12P 7/40; C12N 1/21

(52) U.S. Cl. 435/136; 435/252.3

(57) ABSTRACT

In the production of genetically-modified, non-pathogenic organisms particularly an improved production and isolation of trans-dihydroxycyclohexadiene carboxylic acids and/or derivatives thereof, the synthesis of the transhydroxy cyclohexadiene carboxylic acids is performed in an enantiomer-pure form without detectable impurities with chorismate by cultivation of at least one genetically altered non-pathogenic organism which has an aromate embolism and has, in comparison with a corresponding, genetically not altered organism, an increased activity of the isochorismate synthase (EntC) and isochorismatase (EntB) and an activity of the 2,3 dihydroxybenzoate synthase (EntA) which is close to zero, or an increased activity of the isochorismatase (EntB) and a reduced activity of the isochorismate synthase (EntC) and an activity of the 2,3-dihydroxybenzoat-synthase (entA) of close to zero.

This is Continuation-In-Part application of International application PCT/EP01/09980 filed Aug. 30, 2001 and claiming the priority of German application 100 42 535.6 filed Aug. 30, 2000.

### BACKGROUND OF THE INVENTION

The invention relates to a method for the improved production and isolation of trans-dihydroxy-cyclohexadienecarboxylic acids and/or derivative products thereof as well as a genetically modified non-pathogenic organism suitable therefor and the use of the trans-Dihydroxy-Cyclo-Hexadiene Carboxylic acids (trans-DCHCs).

Trans-dihydroxy-cyclohexadiene carboxylic acids (trans DCHCs) represent in the framework of the shikimic acid biosynthesis path (FIG. 2) the derivative metabolites of the chorismates. Trans-DCHCs are divided into two constitution isomers (stereoisomers), namely, 2,3-trans-DCHC (corresponding to 5S,6S)-di-hydroxy-cyclohexa-1,3-diene carboxylic acid) and 3,4-trans-DCHC (corresponding to (3R,4R)-dihydroxy-cyclohexa-1,5-dienecarboxylic acid), as shown in FIG. 1. Based on chorismate the 2,3-trans-DCHC is formed by way of intermediate isochorismate. The transformation occurs by way of two enzymes: isochorismate synthase (encoded by the gene entC) and isochorismatase (encoded by the gene entB) 2,3-trans-DCHC is an intermediate product in the biosynthesis of the enterobactin, which is formed in the cell and secreted as a chelating agent when there is an iron deficiency in a cell. The enzyme responsible for breaking down 2,3-trans-DCHC is the 2,3 dihydroxybenzoate synthase (encoded by the gene entA). An overview is presented in FIG. 3. 3,4-trans-DCHC could not be observed so far in prokaryotes. According to current understanding it is formed in small amounts upon over-expression of entB, but has no biological function.

The production of metabolites of the aromatic biosynthesis path and particularly of dihydroxy-cyclo-hexadienecarboxylic acids (trans-DCHCs) on the basis of an enzymatic-fermentative process as well as a pure chemical process is already known. The chemical synthesis however is very complex and has numerous disadvantages described in the literature. It is characteristic for the chemical synthesis that they have no or only a very small enantioselectivity and have a low yield because of the very high aromatization susceptibility of the trans-DCHC. Furthermore, multistage synthesis sequences and generally high reaction temperatures with a high energy input are required for the chemical synthesis. In addition, the chemical syntheses are uneconomical because of the use of poisonous, explosive or environmentally destructive substances and the necessary safety and disposal measures. Below, therefore only the enzymatic-fermentative production is considered. It is noted that for processes the production of trans-dihydroxy cyclohexadiene-carboxylic acids in-vitro as well as in-vivo are known.

Rusnak, F. et al. (Biochemistry, 1990. 29: 1425-1435) and Lin, J. et al. (Biochemistry, 1990, 29: 1417-1425) describe for example, generally NMR-measurements for the in-vitro conversion of isochorismate to 2,3-trans-DCHC with purified isochorismate synthase (entC) or, respectively, isochorismatase (EntB). The in-vitro production of trans-DCHC based on chorismate with the aid of cell extracts from E. coli strains, which were mutated either in the genes entA, entB or entC is described by Young, I. G. et al. (J. Bacteriology, 1971, 106: 51-57). The in vitro conversion of a given chorimate to 2,3 trans-DCHC by means of a cell extract from klebsiella strains is described by Young, I. G. et al. (Biochimica et Biophysica Acta, 1969, 177: 381-388 and Biochimica et Biophysica Acta, 1969, 177: 401-411). It is however a disadvantage of the methods described that the cell extracts were stable for only a short time. And it is even a substantially greater disadvantage that chorismate as a base substrate is available in only small quantities and is very expensive (about 700 Dollar/gram).

An enzymatic fermentation synthesis of chorismate, as pre-stage of the trans-DCHC, with the aid of a living system wherein the E. coli strains used have auxotrophies for the aromatic amino acids, is described by Grisostomi, G. et al. (Bioorganic Chemistry, 1997, 25: 297-305). However only small quantities of chorismate (520 mg) were produced. Because of the instability of the chorismate, further processing results in high losses. The in vivo production of another pre-stage of trans-DCHC, namely of isochorismate with klebsiella pneumoniae is described by Schmidt, K. et al. (Biotechnol. Bioeng. 1995. 45: 285-291). With this process, only 90 mg/l isochorismate is formed. Isochorismate has an even greater instability than chorismate so that also this process is not suitable for providing larger amounts of pre-stages or base components for the production of trans-DCHC and its derivative products.

Müller, R. et al. (Microbiology, 1996, 142: 1005-1012) describes the excretion of trans-DCHC by an in vivo system, that is from a Klebsiella pneumoniae strain, which is potentially pathogenic for humans. These strains have a number of different disadvantages. For example, because of various point mutations, the Klebsiella-pneumoniae strains have auxotrophies for the three aromatic acids phenylalanine, tyrosine and tryptophan, so that these essential amino acids must be added to the culture medium. Furthermore, because of the point mutation, the organism has a tendency to a relatively rapid spontaneous back-mutation, as a result of which it is genetically relatively unstable and not suitable for large-scale industrial fermentation processes. In addition, the Klebsielly strains included heterologous genes of E. coli, specifically the gene entB/entC or only entB (Gibson, M. I. et al.; Biochem. J., 1964, 90:248-256). A change of the entA gene for these strains is not disclosed. With the process disclosed herein, it is however particularly disadvantageous that the desired trans-DCHC products are not exclusively formed but are contaminated by various metabolites, particularly chorismate. Besides chorimates, about 90 mg/l 2,3-trans-DCHC could be isolated by over-expression of entB/entC and about 70 mg/l 3,4 trans-DCHC could be isolated from the culture by over-expression of entB. However, it is difficult to isolate and purify such mixtures and it requires additional time and expenses and results in further losses of the desired product. Also, the bacterium Klebsiella pneumoniae is pathogenic, that is, it is potentially pathogenic to humans and subjected to the safety requirements of the risk group 2. The industrial use of pathogenic or potentially pathogenic organisms for the manufacture of biotechnological products is highly problematic because of high production technological safety requirements and a low market acceptance, particularly in the production of medicines.

Furthermore, in addition to the actual synthesis of trans-DCHC, also the isolation of the desired compounds from the respective production preparation must be ensured with satisfactory yields and a particular selectivity for efficiency reasons. Problematic in this connection is particularly the contamination of the production preparation with similar compounds such as chorismate. Processes with lower selectivity such as liquid-liquid extraction or adsorption/elution, for example ion chromatography, are not suitable in this case. Furthermore, the high sensitivity of the trans-DCHC with respect to acidic, caustic and heat influences limits the separation procedures usable for the product purification.

Also, the reactive extraction/re-extraction with anionic or cationic carriers represents a general reprocessing method, which is used so far however mainly for the separation of heavy metal salts. Examples for the use of the reactive-extraction for the product isolation in biotechnological processes and also the concentration of trans-DCHC by extraction are described (Müller, R. et al., Microbiology, 1996, 142: 1005-1012). All these processes however are designed to provide the respective product in high purity for analytical purposes. This means that the known methods are not suitable for the preparative isolation of trans-DCHC from large volume industrial production preparations.

It is the object of the present invention to provide an improved method for the production and isolation of trans-dihydroxy-cyclohexadienecarboxylic acids, which has satisfactory yields of pure trans-DCHCs and which does not suffer from the disadvantages mentioned above.

### SUMMARY OF THE INVENTION

In the production of genetically-modified, non-pathogenic organisms particularly an improved production and isolation of trans-dihydroxycyclohexadiene carboxylic acids and/or derivatives thereof, the synthesis of the transhydroxycyclohexadiene carboxylic acids is performed in an enantiomer-pure form without detectable impurities with chorismate by cultivation of at least one genetically changed non-pathogenic organism which has an aromate embolism and has, in comparison with a corresponding, genetically not altered organism, an increased activity of the isochorismate synthase (EntC) and isochorismatase (EntB) and an activity of the 2,3 dihydroxybenzoate synthase (EntA) which is close to zero, or an increased activity of the isochorismatase (EntB) and a reduced activity of the isochorismate synthase (EntC) and an activity of the 2,3-dihydroxy-benzoat-synthase (entA) of close to zero.

In this context, the method according to the invention includes also the activities of isoenzymes of the isochorismatase and/or isochorisomate synthase, which have been changed accordingly. It particularly comprises the isoenzyme of the isochorismate synthase (EntC), which is designated MenF and coded by the gene menF (Dahm, C. et al., Biochimica et Biophysica Acta, 1998, 1425: 377-386). Under isoenzymes, enzymes with identical or comparable substrate- and effect specificity are to be understood, which however have different primary structures. The explanations of the present invention apply therefore equally to EntC and MenF and possibly to further iso-enzymes of the isochorismate synthase, even if only EntC is explicitly mentioned for clarity reasons.

An advantage of the method according to the invention is the production of enantiomer-free trans-DCHC utilizing the genetically modified non-pathogenic organisms described earlier. Furthermore, with the method according to the invention, the trans-dihydroxycyclohexadiene carboxylic acids are produced without objectionable side products of similar structure, that is, without impurities such as chorismate and/or isochorismate released from the organism into an aqueous permeate (culture medium). Another important advantage of the present invention is that the trans-dihydroxycyclohexadienecarboxylic acid is isolated from the aqueous permeate in a single-stage process, that is, in a single step without the need for earlier purification steps such as HPLC. Examples for single stage isolation processes suitable in accordance with the invention are the reactive extraction by means of a cationic carrier or the adsorption/desorption in an anion exchanger or an extraction (for example, liquid-liquid extraction at pH 1-3). In this way, in accordance with the invention and in an advantageous manner, high product purities of enantiomer-pure trans-DCHC of at least 90% are achieved.

The method according to the invention comprises also processes wherein the desired products are manufactured and isolated not only successively but which can also be produced and isolated in an integrated procedure for example in the framework of a continuously operated fermentation plant wherein the products are at the same time continuously separated.

In accordance with the invention, non-pathogenic organisms, preferably bacteria, yeasts, fungi and/or plants are employed with the method mentioned above to which at most the safety requirements of the risk group 1 apply (defined by the Genetic Engineering Act, 1990 BGB1 [Official Federal Law Gazette] I 1080; 1993 BGB1 I 2066; 1993 BGB1 III 2121-60-1; 1997 BGB1 I 2390), and which have an individual way of aromatic biosynthesis or into which the information regarding heterologous aromate-biosynthesis procedures have been entered by recombinant DNA methods. This is to be understood as meaning the insertion of the aromatic biosynthesis genes which may be, for example, plasmide-encoded and which can be transferred into the chromosome of the host cells by customary methods such as transformation, conjugation or electroporation.

In accordance with the invention, bacteria are preferred, particularly bacteria of the genus enterobacteria, especially Escherichia coli. Preferred representatives are: E. coli MC4100 (ATCC 35695, DSM 6574) (M. J. Casadaban, J. Mol. Biol. 1976, 104, 541-555 and M. J. Casadaban, S. N. Cohen, Proc. Nat. Acad. Sci. USA 1979, 76, 4530-4533, also in Y. Komeda, T. Iino, J. Bacteriol. 1979, 139, 721-729), E. coli H1882, which corresponds to the strain MC 4100 and is additionally characterized by a dihydroxybenzoate synthase deficiency (entA-deletion; A (fepA-ent)) or E. coli AN193 (G. B. Cox et al., J. Bacteriol, 1970, 104, 219-226 and in B. A. Ozenberger, T. J. Brickman, M. A. McIntosh, J. Bacteriol. 1989, 171 775-783), which also represents an entA-negative mutant. However, this selection is not limiting for the present invention.

The trans-DCHC is produced in accordance with the invention preferably on the basis of carbohydrates especially on the basis of glucose and/or D galactose and or glycerol as carbon source. This particularly applies to the microbial manufacture of trans-DCHC. D-galactose and/or glycerol do not need to be used as primary C-source; they may be added during the fermentation already in progress that is they may be added during growth in a dosed manner. The production according to the invention of trans-DCHC based renewable C-sources is particularly advantageous because of an enormous cost reduction in comparison with the conventional processes which utilize the extremely expensive chorismate as precursor.

Furthermore, with the method according to the invention the titer of an enantiomer-pure trans-dihydroxycyclohexadiene carboxylic acids is increased in comparison with an organism which is not genetically modified by the factor 10-30 already with a small or, preferably, non-existent activity of the 2,3 dihydroxybenzoate synthase (entA). Herein, the low, or nonexistent 2,3 dihydroxybenzoate synthase activity is caused by a partial or complete, preferably 1-99%, especially a 5-90% inactivation of the entA genes for example by deletion of the complete gene or parts thereof. Preferred are so-called entA-negative mutants (entA).

In a preferred variant of the present invention, the above result is achieved for enantiomer-pure trans-dihydroxycyclohexadiene carboxylic acids with an E. coli strain using glucose as a C source. It is furthermore a special advantage of the method according to the invention that, in the E. coli strains according to the invention, no byproducts in the form of chorismate and/or isochorismate are formed, that is, there are no impurities with structurally similar compounds such as the chorismate and/or isochorismate in the desired products, or they are below the detection limit. Under the detection limit in the sense of the present invention a concentration of adverse compounds of similar structure in the range of 0.5 ml/l is to be understood. That is that the chorismate and/or isochorismate in the form of adverse side products are present in concentrations of less than 0.5 mg/l fermentation medium.

In another preferred embodiment of the present invention, a genetically altered E. coli strain is used, whose entA-gene is inactive (entA′) and whose genes entB and entC are overexpressed, whereby a very high yield of 2,3-trans-DCHC in the range of several grams per liter, that is, at least 5 g/l is achieved. In this connection, the culture medium according to Pan, J. et al. (Biotechnol. Lett., 1987, 9: 89-94) is optimized with regard to its ammonia concentration. The preferred strains have, with respect to the corresponding genetically non-altered organisms, increased activities of the enzymes isochorismate synthase and isochorismathase and, ideally, a 2,3 dihydroxybenzoate synthase activity of zero or almost zero.

For better understanding of the invention below several enzyme activities are indicated by actual measurement values. These presentations are given as examples and must not be considered as limiting the present invention. They are measurement values, which were obtained with the non-pathogenic E. coli strain AN193 which is considered the “control strain”. This strain, E. coli AN193 is described by G. B. Cox et al., J. Bacteriol. 1970, 104, 219-226 and in B. A. Ozenberger, T. J. Brickman, M. A. McIntosh, J. Bacteriol. 1989, 171, 775-783. It has a specific enzyme activity of the isochorismate synthase (chromosomally encoded by entC) of about 0.10 U/mg protein and of the isochorismatase (chromosomally encoded by entB) of about 0.012 U/mg protein. By over-expression of the entB-gene a comparably high activity of about 0.29 V/mg protein and by over-expression of the entc-gene a comparably high activity of about 1.17 U/mg protein was achieved.

Surprisingly, the over-expression of entB alone in the E. coli (entA)-strain, with an unaltered isochorismate synthase activity does not result in an excretion of 3,4-trans-DCHC. This was not to be expected particularly in view of the known invetigations using pathogenic Klebsiella pneumoniae strains (Müller, R. et al., Microbiology, 1996, 142: 1005-1012).

It is furthermore surprising that in a further embodiment of the invention using an E. coli strain, which ideally has no or only a small activity of the 2,3-dihydroxybenzoate synthase (EntA), of almost zero (entA-mutant) and an increased activity of the isocharismatase (EntB) and a reduced activity of the isochoriomate synthase (EntC), the excretion of 3,4-trans-DCHC in enantiomer-pure form could be achieved.

The method according to the invention has especially the advantage that, for the chiral trans-dihydroxycyclohexadiene carbolic acids, in each case, an enantiomer-purity (ee) in the area of ≧99.9 can be achieved. No adverse contamination by chorismate and/or isochorismate, that is by side products of similar structure, are detectable wherein the detection limit is in the area of less than 0.5 mg/l.

A small or no activity of the 2, 3 dihydroxybenzoate synthase (EntA) and/or changed activities of the isochorismatase (EntB) or, respectively, isochrorismate synthase (EntC) can be caused by changes on a transcriptional, translational and/or post-translational basis. Those may be, for example, changes of regulatory elements such as the promoter activities, the stability of the mRNAs formed or the proteins with respect to a degradation by nucleases or proteins or the initiation of the translation by ribosome compounds or the reduction of the catalytic enzyme activity itself or by a changed binding behavior or regulators or others. Furthermore, there may also be a very small or non-existent activity of the 2,3 dihydroxybenzoatesynthase by the inactivation of the entA-gene which is preferred in accordance with the invention. This can be achieved by different recombinant DNA approaches which are known as such, for example, insertion or deletion mutagenesis or chemical treatment of the cells with mutagenic agents. Preferred are entA-deletion mutants. The methods mentioned above are only intended to facilitate an understanding of the invention; they should not have any limiting effects.

Increased activities of the enzymes isochorismatase and, respectively, isochorismate synthase may also be achieved by an over-expression of the genes entB/entC for example by an increased number of copies of the gene either jointly in a suitable vector or separately in different vectors, which are suitable and compatible with each other. However, also other procedures may be utilized such as the expression under the control of a strong and/or inducable promoter or the stabilization of the mRNA formed and/or the corresponding proteins with respect to nucleases and/or proteases. As promoters in principle all promoters are suitable on the basis of which an expression in the respective host organism is possible, into which the gene construct is inserted. As an example, an IPTG-induced promoter is mentioned.

The activity of the isochorismate synthase can be reduced by a changed expression of the entc-gene, that is, on a transcriptional or translational basis or by an altered activity of the enzyme already formed (post-translational), for example, by the addition of specific effectors (inhibitors) of the isochorismathase.

A reduction of the gene expression, particularly the entC-expression may require actions on a chromosomal and/or plasmide-encoded basis. In this case, the expression of a gene can be controlled by the activity of the promoter to which the gene is subjected. Here, in accordance with the invention, a promoter is preferred which is “leaky” in its activity, that is, which can be regulated in such a way that the gene expression is greatly reduced, but ideally, not fully eliminated. “Leaky” therefore indicates a promoter activity, which is not fully switched off but permits a small residual activity and, therefore, “leaks”, that is, it is slightly permeable. The expert in the field is familiar with various possibilities for fine regulation of promoters. Furthermore, also, the use and/or the genetic change of other elements, which are effective as regulators such as enhancers or silencers, is possible in accordance with the invention.

Furthermore, for the regulation of the entc gene expression, also a repression of the gene expression by blocking the mRNA formed by means of anti-sense-RNA is conceivable. Procedures resulting in a so-called anti-sense repression are known to the person skilled in the art. Subject of the invention are therefore also the gene constructs and/or actors for achieving the changed gene or, respectively, enzyme activities described above. In the context of the present invention a gene construct includes at least one coding nucleotide sequence of the genes entB and/or entC and/or, if applicable, entA as well as regulatory nucleotide sequences, which are operatively linked to these genes, such as promoters, enhancers, silencers, transcription terminators, targeting sequences (transit signal sequences) and/or translational amplifiers. Vectors in the context of the present invention include additional nucleotide sequences for the selection of transformed host cells and/or for the replication within the host cell or for the integration into the respective host cell-genome. The production of these constructs and vectors are known to the person skilled in the art and is therefore not described in detail. As mentioned already earlier, included herein are also constructs and/or vectors which contain nucleotide sequences coding for the isochorisate mutase and/or isochorismate synthase and/or the isoenzymes thereof, for example, menF.

The method according to the invention is further characterized in that the trans-DCHC formed are almost quantitatively isolated from the permeate (aqueous culture medium) by reactive extraction/re-extraction. Taking into consideration, the product properties in a neutral aqueous solution, this isolation occurs with the aid of cationic carriers, i.e. anion-selective carriers. Herein, the trans-DCHCs, which are present in an aqueous (culture) solution bind to a cationic carrier by means of ionic interaction. As carriers ammonium derivatives are preferred. Particularly preferred is trioctylmethylammonium chloride (Aliquat® 336, Aldrich Chemicals Co.). A possibly occurring toxic effect of the carrier on the bio-system is counteracted particularly in an integrated production and isolation process in that the concentration equilibrium of the carrier in the aqueous and the organic phase is so controlled that the carrier concentration in the aqueous phase of the culture medium is maintained under the toxicity limit critical for the biological system. The procedures available herefor are known to the expert. In accordance with the invention cationic carriers and particularly ammonium derivatives are preferred, which, by sterically exacting substituents, do not permeate or permeate only in small amounts through membranes and which, for that reason do not exceed the toxicity limit for the biological system, that is, the non-pathogenic organism. The membrane permeability is the passage of the carrier through the cell membrane into the biological system, which may occur passively by diffusion or actively by transport processes (for example, symport and/or antiport).

In accordance with the invention also more complex anion selective compounds may form carriers for example from the group of the cyclodextrin-cations, endrohedral-cationic fullerenes, cationically charged crown ethers and/or kryptands.

In accordance with the invention, during the reactive extraction, cationic carriers are employed in a concentration of 1-30 Vol %, preferably 3-20 vol %, especially 5-15 vol % based on the culture medium. The extraction volume increases almost proportionally with the carrier concentration used. With the method according to the invention, oxygen-containing solvents of a medium chain number of C₄ to C₂₀, preferably C₆ to C₁₂ such as 2-undecanon and/or diphenyl ether and/or butylbenzene and/or 1-octanol are used as organic solvents wherein 1-octanol is particularly preferred.

As uncharged and essentially non-polar trans-DCHC/carrier ion pair both compounds are transferred into a non-polar organic phase. From this organic phase, the 2,3-trans-DCHCs are again extracted by a third phase (pure phase) with high counter-ion concentration. The driving force for the enrichment of the products in the pure phase is the high concentration difference of the anti-ions. No negative effects of an irreversible product bonding to the carrier was observed, so that the trans-DCHC can be re-extracted from the organic phase almost quantitatively.

With the reactive extraction, as re-extraction means saturated inorganic salt solutions, preferably with chloride- and/or carbonate ions, particularly in the form of a saturated sodium chloride solution, are used. Furthermore with the method according to the invention, the re-extraction performance increases proportionally with the anion concentration and there is no direct dependency on the pH value.

It has further been found to be advantageous to liberate the reextracted trans-DCHC in the pure-phase from water by extraction with organic solvents without the use of aqueous carriers and then to concentrate them by distillation. In this procedure, no losses occur if it is performed at room temperature and with an acid pH value and if no catalytically active surfaces such as silica gels are used. With the method according to the invention, the extraction of the enantiomer-pure trans-dihydroxycyclohexadiene carboxylic acid from the salt load occurs almost quantitatively with organic solvents such as medium-chain alcohols, volatile carbon acid esters, ketones and/or functionalized aromatic compounds, at an acid pH value of pH 0 to pH 5, preferably at pH 3. Examples for preferred organic solvents are various acetic esters, acetone, diphenyl ether and/or 1-butanol, the latter being preferred in accordance with the invention. The examples serve as explanation for the invention and are not limiting. A schematic overview of the purification of the trans-DCHC according to the invention is presented in FIG. 4.

A particular advantage of the method according to the invention and here especially with the reactive extraction/reextraction is that the trans-DCHCs are processed under very mild conditions so that the products are not aromatized.

In another embodiment, the trans-DCHCs are isolated from the aqueous permeate (culture solution) by adsorption/desorption on an ion exchanger, preferably an ion exchanger such as the resin DOWEX 1×8 (100-200 mesh). Then the culture solution can be applied directly, that is, without further processing, to a fluidized bed or a solid bed or suitable adsorption particles. Under certain circumstances, the particulate (solid) substances first have to be separated, particularly with an adsorption in a fluidized bed. Independently of such an initial separation of particulate components, the culture solution to be deposited on the adsorber is adjusted to a pH value in the alkaline range preferably to a pH value in the range of 7.5-11, especially to a pH of 8. The conductivity of the solution is adjusted by suitable measures to a value of 2.5-20 mS/cm, preferably 8-15 mS/cm, especially 13.5 mS/cm. It is particularly advantageous with this embodiment of the method according to the invention, that it provides for a high selectivity and the trans-DCHC formed during the fermentation and transferred to the aqueous permeate are surprisingly collected on the adsorber as the main components. The trans-DCHC are desorbed for example by washing of the adsorber with aqueous solutions with an acid pH value. Preferred are volatile acids, with formic acid being particularly preferred. For the isolation of the trans-DCHC in solid form a freeze-drying procedure may be used. Surprisingly, the trans-DCHC remain mainly as solid residue with the use of the desorption means mentioned above. With the procedure described above product purities of the trans-DCHC of at least 90% are achieved.

Subject of the present invention is also a genetically changed organism for the improved manufacture of trans-dihydroxycyclohexadiene carboxylic acids in an enantiomer-pure form without detectable impurities of chorismate and/or isochorismate which is characterized in that it is a non-pathogenic organism which is subject at most to the safety requirement of the risk group 1 and, in comparison with a corresponding genetically not altered non-pathogenic organisms, has a 2,3 dihydroxybenzoate synthase activity (EntA) of zero or almost zero (that is, ideally zero) and an increased activity of the isochorismatase (EntB) and an increased or a reduced activity of the isochorismate synthase (EntC).

Herein, an only small or missing activity of the 2,3 dihydroxybenzoate synthase can be achieved in that the corresponding gene entA is inactivated. This can occur non-directionally by classical chemical mutagenese and selection of auxotrophic mutants, or for example, by directed gene technical inactivations (for example, insertions, deletions) of the gene and subsequent selection of clearly defined mutants. Further, the expression on a transcriptional and/or translational basis may be reduced or blocked. Also conceivable is that the catalytic activity on protein basis (post translational) is reduced or the binding behavior of effectors (activators or inhibitors) is changed.

In a special embodiment of the invention, the entA-gene coding for the 2,3 dihydroxybenzoate synthase of the genetically changed organism is deleted and its genes entC and entB encoding for the isochorismate synthase and isochorismatase are expressed in an amplified manner.

The invention therefore comprises a genetically changed organism in which the entA-gene is deleted completely or partially, preferably over a range of at least 1-99% and preferably 5-90% and the entB gene is more strongly expressed and the expression of the entc-gene is, depending on the desired product, increased or reduced. Further, the activity of the isochorismate synthase encoded by entc can be regulated also on a translational and/or post-translational basis. This may be achieved by procedures for the anti-sense repression or retardation with specific effectors.

The invention furthermore resides in a genetically changed organism which contains at least one of the genes entb, entc and homologs thereof wherein the genes may be isolated from homologous or heterologous organisms.

Homologous nucleotide sequences are in accordance with the invention sequences which are complementary to the nucleotide sequences of the used host organism or which hybridize with these nucleotide sequences. The term hybridizing sequences includes in accordance with the invention substantially similar nucleotide sequences from the group of DNA or RNA which, under known stringent conditions, have a specific reaction (binding) with the nucleotide sequences mentioned earlier. The nucleotide sequences which have been entered into the organism according to the invention and which code for the genes entB or entC may of course be naturally occurring sequences as well as artificial sequences, for example, nucleotide sequences obtained by chemical synthesis and possibly nucleotide sequences, which are adapted to the codon use of the host organism. Under homologous organisms such organisms are to be understood which belong to a related family. Correspondingly, heterologous organisms are less closely related organisms.

A genetically altered organism of the type described above forms, by an increased expression of the entC gene, together with the increased expression of the entB gene, in an entA-negative mutant increased amounts of 2,3-trans-dyhydroxycyclohexadiene carboxylic acid.

In another variant, a genetically changed organism forms, by a reduced expression of the entC-gene (and, at the same time increased entB expression in a entA-negative mutant), increased amounts of 3,4 trans-dihydroxycyclohexadiene carboxylic acid. Herein the reduced expression of the entC gene may be achieved for example by a controllable promoter activity, to which the entC-gene is subjected. This promoter acitivity may be “leaky” that is it may be very small but does not need to result in a 100% blockage of the expression. Furthermore, an anti-sense-repression of the entC gene is conceivable. In principle, herewith, based on the coding entC gene sequence, a mRNA is transcribed, which is then hybridized with a corresponding anti-sense mRNA and can therefore not be translated, so that the entC gene is repressed. The corresponding anti-sense mRNA may be formed for example on the basis of a gene construct, which includes the entC gene in anti-sense-orientation under the control of a preferably controllable promoter. This gene construct is produced in accordance with generally known methods of genetic engineering and is transferred into the non-pathogenic genetically altered organism.

Generally, all the regulative elements known as such are covered by the invention, which can control the expression of genes in host cells. This includes besides promoters among others also enhancers or silencers.

Generally, a reduced isochorismate synthase activity may, by a regulation on a post translational basis, result in an increased formation of 3,4-trans-DCHC, for example, by the addition of specific effectors to the culture preparations.

Subject of the present invention is furthermore a genetically changed organism, which includes a gene construct, which has at least one controllable promoter, which is operatively connected at the 5′ end with the entC structure gene. Herein, the entC-structure gene is present in the so-called “sense” orientation. The invention covers also a genetically changed organism, which includes a gene construct that itself contains a preferably controllable promoter, which is operatively connected to an entC-nucleotide sequence in “anti-sense” orientation.

An operative connection is to be understood to be a sequential arrangement for example of promoter, coding sequence, terminator and possibly of further regulatory elements in such a way that each of the regulatory elements can fulfill its function during the expression of the coding sequence as predetermined. These regulatory nucleotide sequences may be of natural origin or they may have been obtained by chemical synthesis. As promoter, basically any promoter is suitable which can control the gene expression in the respective host organism. In accordance with the invention, this maybe a chemically inducible promoter, by which the expression of its subordinate genes in the host cell can be controlled at a certain point in time. An example herefor is a promoter inducible by IPTG (Isopropyl-β-thiogalactoside).

A gene structure is produced by fusion of a suitable promoter with at least one nucleotide sequence in accordance with common recombination and cloning techniques as they are described for example in Sambrook, J. et al., Molecular Cloning: A Laboratory Manual, Cold Spring Harbor Laboratory, Cold Spring Harbor, N.Y. (1989). For the connection of the DNA fragments to one another, adapters or linkers may be attached to the fragments.

The present invention further resides in a vector, which includes at least one nucleotide sequence of the type described above, regulative nucleotide sequences operatively connected thereto and additional nucleotide sequences for the selection of transformed host cells, for the replication within the host cells or for the integration into the respective host cell genome. The vector according to the invention may also include a gene structure of the type described above.

The genetic changes of the organism according to the invention may be present in chromosomal and/or extra-chromosomal and then preferably in a plasmid-encoded form.

Subject of the invention are further enantiomer-pure trans-dihydroxycyclohexadiene carboxylic acids produced by a method of the type described above, which have an acid constant pKₛ in the range of 3 to 5 and which are very stable at room temperature and a pH value in the range of 4 to 13, preferably 5 to 11 with a disintegration constant of less than 0.1 d⁻¹ corresponding to a half-life of at least 7 days.

The invention also resides in the use of the respective enantiomer-pure trans-dihydroxycyclohexadiene carboxylic acid for the production of complex biologically active metabolism metabolites, for example plant metabolites, particularly cyclohexandiol epoxides and/or of carbohydrate mimetics, especially carba sugar and specifically amino carba sugar.

The use of cis-dihydroxycyclohexadiene carboxylic acid is also known. However, the formation of the cis-DCHC is based on an oxidative dioxygenation of aromatic compounds, which must be added to the medium in a fermentative manufacturing process. With the fermentative preparation according to the present invention of enantiomer-pure trans-DCHC with high yields, substantially more economical possibilities and in some ways completely new directions for the synthesis of natural and other effective substances become available.

The trans-DCHC according to the invention may be used for example as starter compounds for the preparation of derivatives of biologically active metabolism metabolites, for example, plant metabolites. Characteristic for the substance class of these metabolites is the 2,3 dihydroxycyclohexylmethanol base structure, which is epoxidized at the 4-,5-, or 6 position. Best known representatives are, among others, crotepoxide, isocrotepoxide, epicrotepoxide, senepoxide, β-senepoxide, pipoxide, cyclophallitol, boesenoxide and tingtanoxide, which are represented in FIG. 7.

For these classes of substances, it could be shown that, as carbohydrate mimetics, they have inhibitory effects, for example, tumor inhibition effects with regard to various carcinomas and therefore are potentially suitable for pharmaceutical applications. They are being produced by expensive chemical synthesis with the known disadvantages of high costs and time consumption, and small enantioselectivity to name just the most important. With these synthesis also derivatives of the trans-DCHC occur as intermediate compounds. An overview of the most important syntheses of the cyclohexandiol epoxide plant-metabolites, in which a 2,3 trans-DCHC derivative represents, in each case, a key substance, is shown in FIG. 8. Providing the trans-DCHC facilitates therefore another field of application for these compounds and therefore contributes to an essential simplification of the expensive synthesis of complex natural compounds and effective substances since the synthesis may now be based directly on the fermentatively produced trans-DCHC and expensive synthesis and purfication steps for the preparation of the trans-DCHC are no longer necessary. The present invention therefore has an enormous economical potential.

Based on the trans-DCHC according to the invention also the synthesis of carba sugars and particularly amino carba sugars is possible which also have a high pharmaceutical potential as carbohydrate mimetics. Important representatives of this class of substances are for example valienamines (FIG. 9).

Valienamine derivatives are of high interest as base compounds for the synthesis of carbohydrate mimetics such as a carbose or voglibose, which are of great interest among others as α-glucosidase inhibitors for the abatement of age-based diabetes or of obesity.

The present invention also resides in the use of the trans-DCHC (for example, bound to a polymer carrier) for the identification and/or preparation of other effective substances such as polysaccharid-mimetics and/or for the establishment of substance libraries for example for the so-called “high throughput screening” experiments.

Another possible application of the trans-DCHC according to the invention is a large spectrum of biologically active substances, such as the effective substance class of the immunosuppressives or anti-tumor drugs to mention only a few. A selection of such compounds is presented in FIG. 10.

Furthermore, on the basis of the trans-DCHC according to the invention, a multitude of complex compounds can be produced, which are designated here generally as follow-up chemistry. In this connection, particularly a predetermined reaction control for example by a selective reduction of the carboxyl group, a selective epoxidizing of the double bonds and/or other follow-up modifications are possible. Possible follow-up chemical compounds are presented in FIG. 11 on the basis of the example of the 2,3-trans-DCHC as base substance.

Examples of the invention are presented below in order to illustrate the invention. They should not have any limiting effects.

1. General Genetic Techniques:

The isolation of genomic DNA and plasmid DNA and all the techniques for the restriction, Klenow- and alkaline phosphatase treatment, also methods for the cloning, sequencing and amplifying (PCR) of DNA as well as the transformation, cultivation and selection of host cells were performed in accordance with Sambrook, J. et al., (Molecular Cloning: A Laboratory Manual, Cold Spring Harbor Laboratory, Cold Spring Harbor, N.Y., 1998.

2. Isolation of the entB and entC Genes and Cloning them Into the Expression Vector pJF 119EH1:

The entB and entC genes were isolated from the E. coli strain w3110 (J. Lederberg, Yale University, USA). A description of this wild strain is found in C. W. Hill, B. W. Harnish, Proc. Natl. Acad. Sci. USA, 1981, 78 7069-7072. The chromosomal DNA was prepared in accordance with a standard protocol obtained from R. Sussmund, J. Eberspacher, R. Hagg, W. Springer, Mikrobiologisch-Biochemisches Praktikum, 2. Edition Thieme Verlag, Stuttgart, New York 1999. From chromosomal DNA entB was amplified by means of PCR. The oligonucleotides used were TAT GGA TCC ACG ATC AGC CTG AA and GGG CTG CAG ACA TTT TTA CCG CTG. The PCR conditions were: denaturizing 5 min (94° C.), 34 cycles:—denaturizing 4 min (94° C.), annealing 2 min (57° C.), prolonging 1.5 min (75° C.)—, holding 7 min (75° C.), cooling to 4° C. The amplified entB-fragment (855 bp) was isolated by gel electrophoresis (0.8% agarose gel, ethidiumbromide-detection) and purified by affinity chromatography (Qiagen extraction Kid, Qiagen). The fragment was mixed with the endonucleases BamH1 and Pstl and purified by ion exchange chromatography (midi-Kid, Qiagen).

The gene entC was generated in a corresponding way. With chromosomal DNA from E. coli W3110 a PCR with the oligonucleotides GGC GAG CTC ATT ATT AAA GCC TTT AND TGC GGA TCC TCG CTC CTT ATT GC was performed. The PCR-conditions were: 2 min (94° C.), 10 cycles:—denaturing 0.5 min (94° C.), annealing 0.75 min (40° C.), extending 1 min (72° C.)—, 20 cycles;—denaturing 0.5 min (94° C.), annealing 0.75 min (55° C.), prolonging 1 min+10 sec/cycle (72° C.)—, cooling to 4° C.

The DNA product (1220 bp) was, like entB, electrophoretically purified and extracted. The product was mixed with the endonucleases BamH1 and Sacl and purified by ion exchange chromatography.

Construction of the Plasmids pDF1 and pDF2.

The entB gene was ligated with T4 DNA ligase into the mixed vector pJF119EH1(Fürste, J. P. et al., Gene, 1986, 48: 119-131) (section locations BamH1, Pstl). The plasmid was transformed into the cloning and expression strain E. coli DH5α and subsequently cultivated on agar plates (LB, 100 mg/1 ampicillin) to form individual colonies. The plasmid pDF1 (pJF119EH1 with insert entB) was selected and verified by a restriction analysis.

In an analogous manner, the entC-gene was inserted into the cut vector pDF1 (cleavage sites Pstl, Sacl). The DNA product was verified again by restriction analysis. With both plasmids a sequencing along the inserted genes was performed. The plasmid construction is illustrated in FIG. 5.

The plasmids were transformed into the E. coli strains AN193 and H1882. The expression of active proteins was confirmed by SDS-PAGE and enzyme activity tests. The E. coli strains used are characterized as follows:

The strain AN193 corresponds to Elter AB1515, but has a number of deletions (trp leu proc lac fhuA rpsL entA403). The strain AN193 is described by G. B. Cox et al., J. Bacteriol. 1970, 104, 219-226 and in B. A. Ozenberger, T. J. Brickman, M. A. McIntosh, J. Bacteriol, 1989. 171, 775-583. The strain H1882 corresponds to the strain MC4100 (DSM 6574, ATCC 35695), but has the dihydroxybenzoate synthase deficiency (Δ(fepA-ent)). A more extensive description of the strain MC4100 is provided by M. J. Casadaban, J. Mol. Biol. 1976, 104, 541-555 and by M. J. Casadaban, S. N. Cohen, Proc. Natl. Acad, Sci USA 1979, 76, 4530-4533 and also by Y. Komeda, T. Iino, J. Bacteriol. 1979, 139, 721-729.

3. Fermentation for the Production of Trans-DCHC

100 μl of a 50% glycerol culture (feed stock) of the strain AN193/pDF2 are used for the inoculation of pre-culture 1 consisting of 5 ml yeast extract medium (yeast extract 5 g/l, casein peptone 10 g/l, NaCl 5 g/l, ampicillin 100 mg/l) in a 20 ml test tube. The cells were cultivated at 37° C. under shaking (150 rpm) for 12 hrs. With 500 μl from pre-culture 1, the medium for pre-culture 2 was inoculated, consisting of 3×100 ml yeast extract medium (yeast extract 5 g/l, casein peptone 10 g/l, NaCl 5 g/l, ampicillin 100 mg/l) in a 1 liter baffled shaking flask. The cells were cultured at 37° C. under shaking (150 rpm) for 12 hrs. Pre-culture 2 was transferred into a 7.5 l aerated and pH-controlled stirred tank reactor containing 5 liter main culture medium (KH₂PO₄ 13 g/l, K₂HPO₄ 10 g/l, NaH₂PO₄2H₂O 6 g/l, (NH₄)₂PO₄ 2 g/l, NH₄Cl 0.2 g/l, MgSO₄7H₂O 3 g/l, trace solution 1 ml/l, ampicillin 100 mg/l, thiamin 20 mg/l, leucin 20 mg/l, prolin 20 mg/l, adenin 20 mg/l, tryptophan 20 mg/l, 2,3-dihydroxy-benzoic acid 20 mg/l, glucose 30 g/l which had been sterilized by filtration.

The trace solution contained dissolved in 5N HCl solution: FeSO₄7H₂O 40 g/l, CaCl₂H₂O 40 g/l, MnSO₄nH₂O 10 g/l, AlCl₃6H₂O 10 g/1, CoCl₂ 4 g/l, ZnSO₄7H₂O 2 g/l, Na₂MoO₄H₂O 2 g/l, CuCl₂2H₂O 1 g/l, H₃BO₃ 0.5 g/l.

The fermentation was aerated by a gas stream of 4.03 l/min. The pH was adjusted with 25% aqueous ammonia solution to 7.0. The temperature was adjusted to 37° C. The stirring speed was 500 rpm. The concentration of dissolved oxygen was adjusted to be above 20% air saturation by controlling the air admission and the stirring speed. Antifoaming agent AF 298 (Sigma) was added as needed. After reaching a dry bio mass of 6 g/l, 1 PTG(132 mg in 5 ml water) was added, which had been sterilized by filtering, for the purpose of induction. After consumption of the start-up glucose, a sterile glucose concentrate (700 g/l) was fed in. The flow volume was 0.17 ml/min. After 50 h processing time, the concentration of 2.3-trans-DCHC was 930mg/l (6mM).

By further optimizing the medium composition, for example, by increasing the ammonia concentration and optimizing the fermentation conditions, particularly the pH value and the temperature, the dry bio mass increased (above 20 g/l BTM). With higher bio dry masses higher product fermentation rates (greater than 60 mgl⁻¹h⁻¹g(BTM)⁻¹) as well as a higher product titer (above 5 g/l for 2,3-trans-DCHC with strain AN193/pDF2) can be achieved. An increase in the bio dry-masses can be achieved especially also in that, after or during the growth phase, instead of glucose, other carbon sources are used for the production of trans-DCHC, particularly D-galactose or glycerin.

The process data of an exemplary fermentation with further improved conditions are shown in FIG. 6.

4. Reactive Extraction of the 2,3-trans-DCHC and Separation of the Salt Charge:

The cationic carrier Aliquat® 336 was dissolved with 5 vol % in the organic solvent 1-octanol at a pH 7 and used for the extraction out of the culture medium of the 2,3-trans-DCHC formed.

The re-extraction of 2,3-trans-DCHC occurred with saturated sodium chloride solution at pH 7.

The pure phase of the reactive extraction contains, in addition to the trans-DCHC, a certain amount of salts in aqueous solution. By extraction with an organic solvent the product can be quantitatively separated from the salt load. This is done with 1-butanol at pH 3, wherein extraction coefficients of at least 30% were achieved. The product, dried with magnesium sulfate over night and then filtered and dried in vacuum, is then free of salts and water. In the 1H-NMR- and 13C-NMR spectrum of the white-yellow solid material no impurities particularly no chorimate and/or isochorismate can be detected.

5. Isolation of the 2,3-trans-DCHC by Adsorption and Desorption.

After the fermentation, solid particular components are removed from 2.2 liters of the culture solution by zentrifuging and the solution is adjusted to a pH value of 8 and a conductivity of 13.5 mS/cm. As adsorber 530 ml DOWEX 1×8 (CL-form, 100-200 mesh) are filled into a column to form a 27 cm sedimentary bed length and an equilibrium is provided with 50 mM dipotassium hydrogen phosphate buffer (pH 8). The adsorber is charged with 2.2 l of the culture solution at 2.4×10⁻⁴ m/s. After charging washing takes place with 5 times the column volume by a 50 mM dipotassium hydrogen phosphate buffer (pH 8) at the same flow speed. Then the desorption step is performed at the same flow speed using 8 times the column volume of 50 mM formic acid. The desorption fraction is then freeze-dried. An analysis by ¹H-NMR and HPLC chromatography of the solid material obtained shows no impurities.

### DESCRIPTION OF THE DRAWINGS

FIG. 1 shows structure formulas of the trans-dihydroxycyclohexadiene carboxylic acid stereoisomer. Shown are (5S,6S)-dihydroxy-cyclohexa-1,3-diene carboxylic acid and (3R,4R)-dihydroxycyclohexy-1,5-diene carboxylic acid.

FIG. 2 is an overview of the shikimate biosyntheses path. Chorismate is the base substance of the biosynthesis of the aromatic amino acids, of the chelating agent enterobactin, the folates the menaquinones and the ubiquinones.

FIG. 3 shows the biosynthesis path of chorismate to 2,3-trans-DCHC (by way of isochorismate) and 3,4-trans-DCHC, etnC coded isochorismate synthase, entB coded isochorismatase, entA coded 2,3 dihydroxybenzoate synthase.

FIG. 4 shows the purification of trans-DCHC by reactive extraction. The organic phase serves as so-called “selective revolving door” for the product extraction. Trans-DCHCs are transported from the aqueous phase as lipophilic ion pair carrier-DCHC through the organic phase into the aqueous product phase. Driving force of the enrichment of the trans-DCHC in the product phase is the concentration difference at the counterions. The charge exchange of the two phases occurs by return transport of the chloride.

FIG. 5 shows schematically the construction of the plasmids PDF1 and pDF2.

FIGS. 6a and 6b show an example of a fermentation of E. coli strain AN193 with plasmid pDF2 for the production of 2,3-trans-DCHC. Shown is the time dependency of the bio dry mass, the glucose concentration and the product titer.

FIG. 7 shows structure formulas of biologically active plant metabolites. All the substances contain the basic structure of the 2,3-dihydroxy cyclohexylmethanol skeleton having a vicinal diol-unit in trans.

FIG. 8 is an overview of the most important synthesis of the cyclo hexandiol-epoxide plant metabolites. Key substance is in each case a derivative of the 2,3-trans-DCHC.

FIG. 9 is an overview of the structure formula of the substance class of the amino carba sugars.

FIG. 10 is an overview of the structure formula of additional target classes.

FIG. 11 is an overview of general structure formulas of target compounds (follow-up chemistry) which, based on the trans-DCHC according to the invention, can be provided in a targeted manner, that is, by selective reduction epoxidation and modification. By way of example, as a starter substance, a 2,3 trans-dihydroxy cyclohexadiene carboxylic acid is shown.

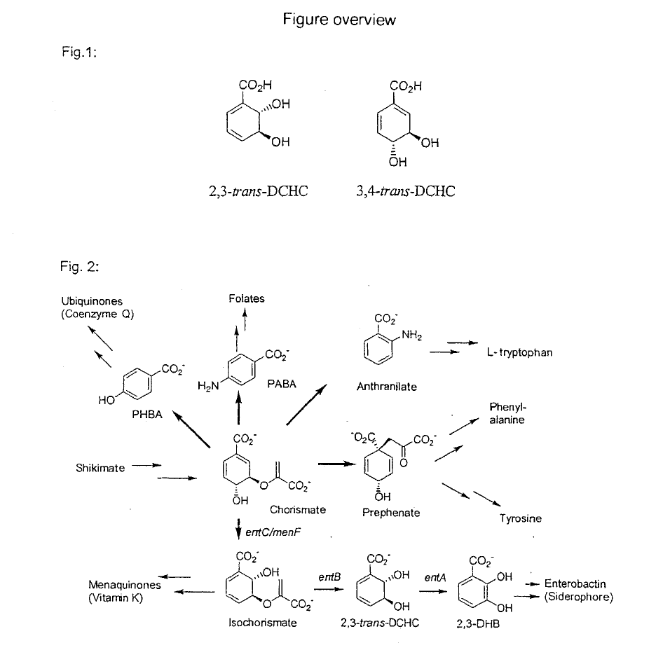

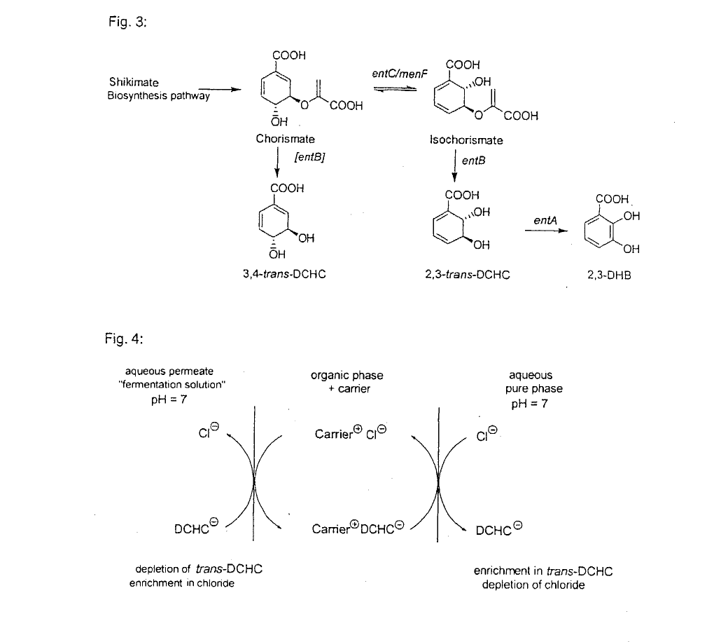

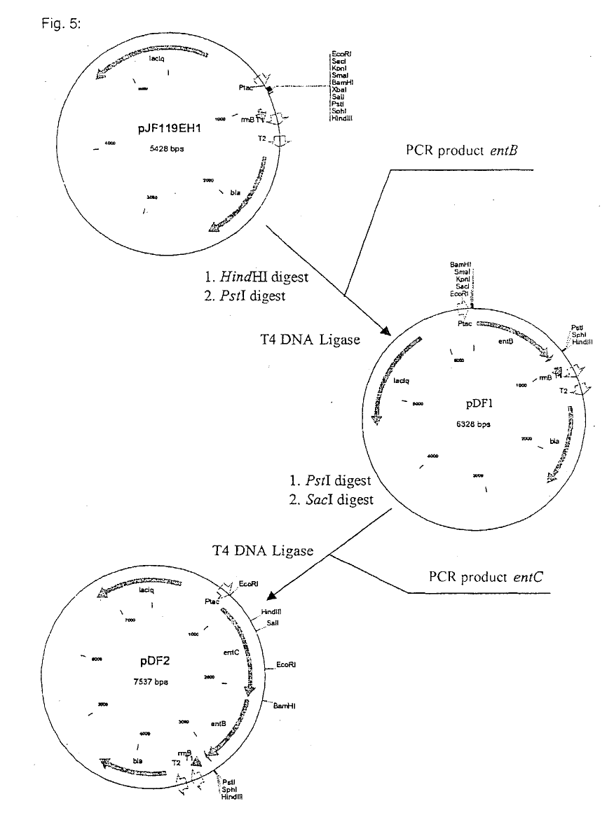

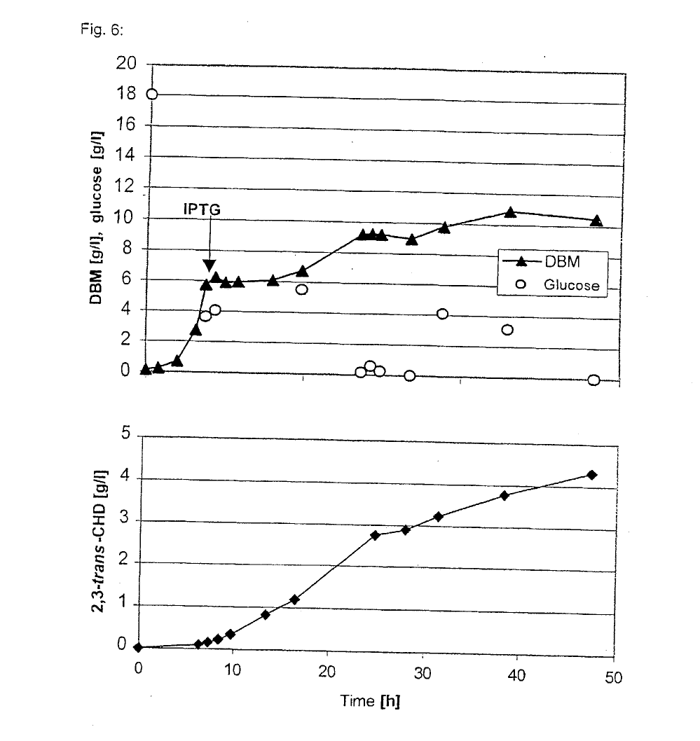

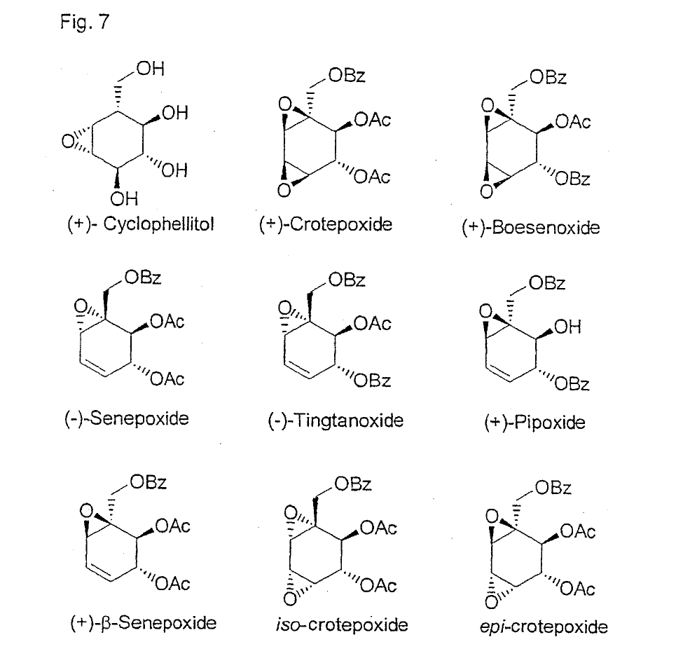

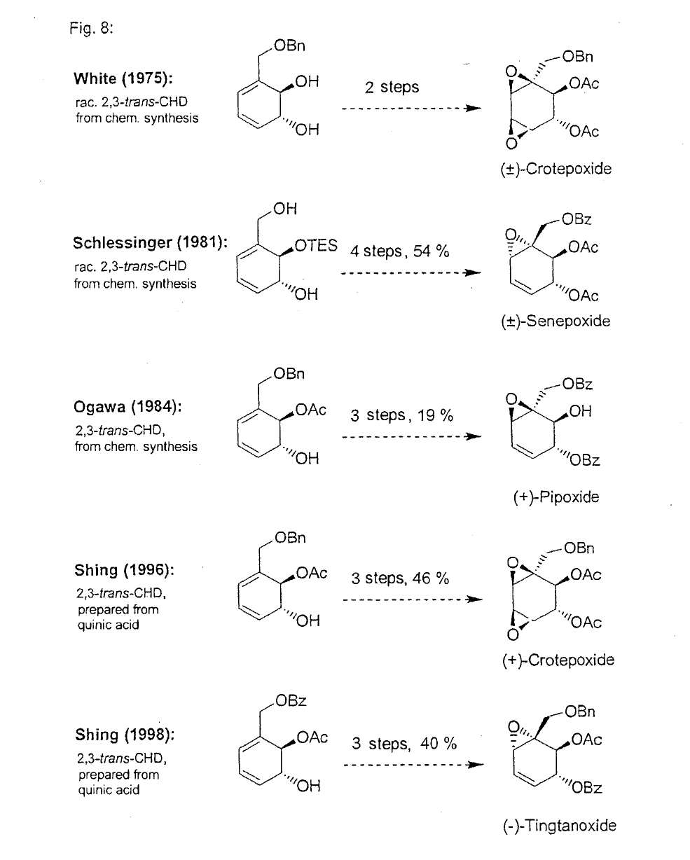

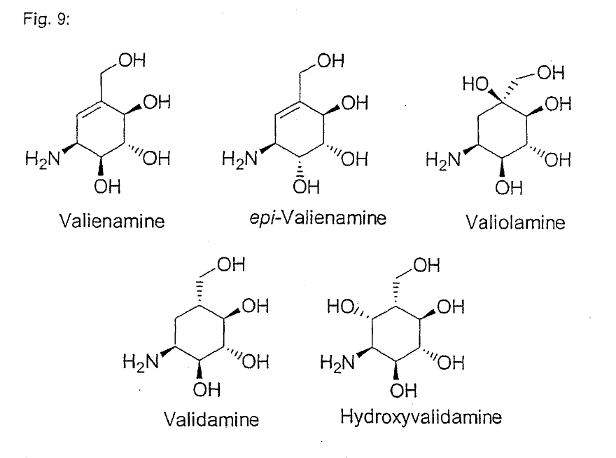

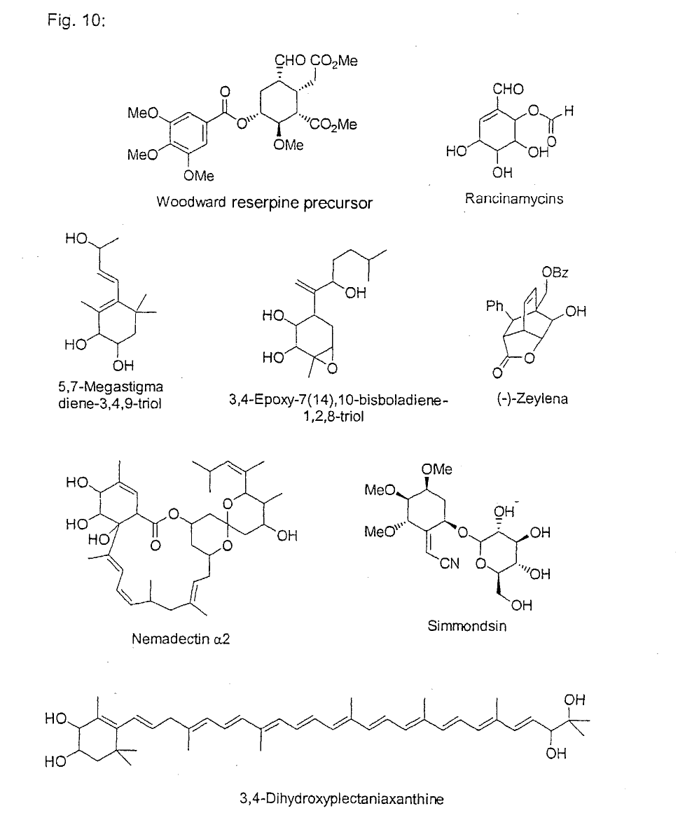

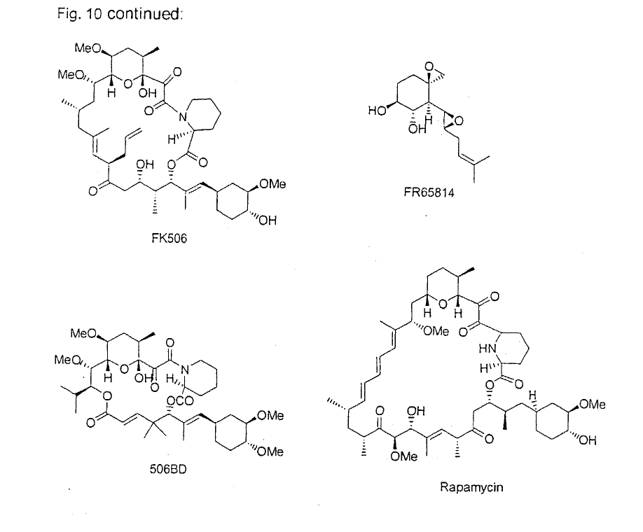

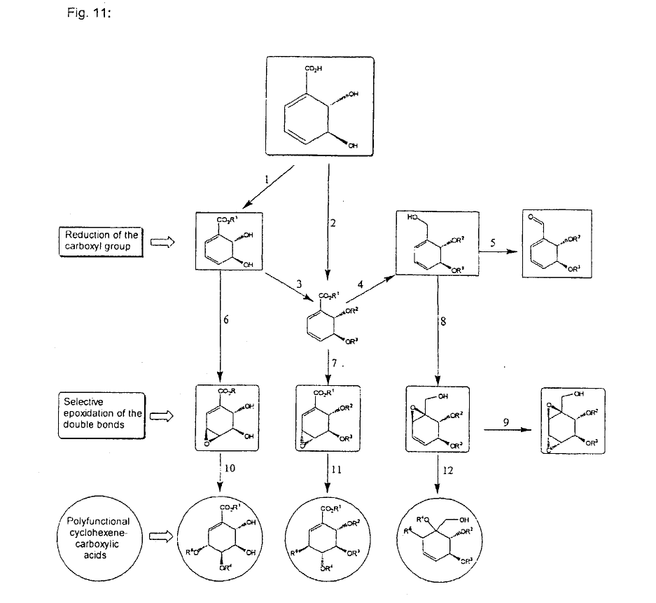

What is claimed is:

1. A method for the improved production of transdihydroxycyclohexadiene carboxylic acid and derivative products thereof, comprising the steps of performing the synthesis of the trans-hydroxycyclohexadiene carboxylic acids in an enantiomer-pure form without detectable impurities with chorismate on the basis of renewable carbon sources by cultivation of at least one genetically altered non-pathogenic organism which has an aromate embolism and has, in comparison with a corresponding, genetically not altered organism, one of
a) an increased activity of the isochorismate synthase (EntC) and isochorismatase (EntB) and an activity of the 2,3 dihydroxybenzoate synthasb (EntA) which is close to zero, and
b) an increased activity of the isochorismatase (EntB) and a reduced activity of the isochorismate synthase (EntC) and an activity of the 2,3-dihydroxybenzoat-synthase (entA) of close to zero.

2. A method according to claim 1, wherein trans-dihydroxycyclohexadiene carboxylic acids transferred from the organism into an aqueous permeate are isolated without additional processing steps from the aqueous permeate by one of a reactive extraction with a cationic carrier and an absorption/desorption on an anion exchanger and a liquid-liquid extraction, whereby enantiomer pure trans-DCHC with product purities of at least 90% are obtained.

3. A method according to claim 1, wherein non-pathogenic organisms comprising at least one of bacteria, yeasts, fungi and plants are used which are subject at most to the safety requirements of the risk group 1.

4. A method according to claim 3, wherein bacteria of the strain entereobacteria, preferably Escheria coli, are used.

5. A method according to claim 1, wherein, with an at least partial inactivation of the gene encoding the 2,3 dihydroxy benzoate synthase (entA) an increase of the titer of enantiomer-pure trans-dihydroxycyclohexdiene carboxylic acids in comparison with a genetically not changed organism by a factor of 10-30 is achieved.

6. A method according to claim 1, wherein for the chiral trans-dihydroxycyclohexadiene carboxylic acids an enantiomer purity (ee) of ≧99.9% is achieved.

7. A method according to claim 2, wherein during the reactive extraction at least one of ammonia derivatives, preferably ammonia derivatives with sterically demanding substitutes and a carrier from the group consisting of cyclodextrin cations, endohedral-cationic fullerenes, cationically charged kronenether and kryptands are used as cationic carrier.

8. A method according to claim 2, wherein, in the reactive extraction, cationic carriers in a concentration in the range of 1-30 vol % preferably 5-15 vol % based on the culture medium are used.

9. A method according to claim 8, wherein during the reactive extraction the extraction performance increases essentially proportionally with the carrier-concentration employed.

10. A method according to claim 8, wherein, during the reactive extraction, as organic solvent an oxygen containing solvent of an average chain number, preferably at least one of 2-undecanon, diphenylether, butyl benzene and 1-octanol is used, with 1-octanol being the preferred solvent.

11. A method according to claim 8, wherein in the reactive extraction as re-extraction means saturated inorganic salt solutions including at least one of chloride- and carbonate ions are employed, with sodium chloride solution being the preferred salt solution.

12. A method according to claim 8, wherein in the reactive extraction the re-extraction performance increases proportional with the anion concentration and there is no direct dependency on the pH value.

13. A method according to claim 8, wherein the extraction of the enantiomer-pure dihydroxy-cyclohexadiene carboxylic acid from the salt load occurs almost quantitatively at an acid pH value in the range of pH 0 to pH 5, preferably at pH 3, using organic solvents including medium-chain alcohols, volatile carboxylic acid esters, ketones and functionalized aromates, preferably one of 1-butanol, acetic ester, acetone and diphenyl ether.

14. A method according to claim 2, wherein the adsorption/desorption is performed in one of a fluidized bed and a stationary bed with preceding removal of any solid materials.

15. A genetically altered organism for the improved production of trans-dihydroxycylohexadiene carboxylic acids in enantiomer pure form without detectable impurities by chorismate, said genetically changed organism being
a) a pathogenic organism which is subjected at most to the safety requirements of the risk group 1 and, in comparison with a corresponding genetically unaltered organism, has
b) a 2,3 dihydroxybenzoate synthase-activity (EntA) of almost zero,
c) has an increased activity of the isochorismatase (EntB) and
d) has one of an increased and reduced activity of the isochorimate synthase (EntC).

16. A genetically altered organism according to claim 15, wherein the entA gene is at least partially deleted, the entB-gene expression is increased and the expression of the entC gene is increased or reduced.

17. A genetically altered organisms according to claim 16, wherein an increased amount of (5S,6S)-dihydroxycyclohexa-1,3-diene carboxylic acid is formed by the increased expression of the entC gene.

18. A genetically altered organism according to claim 16, wherein with a reduced expression of the entC gene, an increased amount of (3R,4R)-dihydroxycyclohexa-1,5-dienecarboxylic acid is formed.

19. A genetically changed organism according to claim 15, wherein said altered organism includes at least one of a gene construct and a vector with at least one promotor which is operatively linked to the entC structure gene.

20. A genetically changed organism according to claim 15, wherein said changed organism includes at least one of a gene construct and a vector with at least one promotor, which is operatively linked to the entC structure gene in an antisense orientation.

21. An enantiomer-pure trans-dihydroxy-cyclohexadiene carboxylic acid produced by a method according to claim 1, with an acid constant pKs in the range of 3 to 5 which, at room temperature and a pH value in the range of 4 to 13, preferably 5 to 11, with a disintegration constant of less than 0.1d⁻¹ corresponding to a half life of at least 7 days, is very stable.

22. The use of the enantiomer-pure transdihydroxycyclohexadiene carboxylic acid according to claim 21 for the production of complex biologically active metabolism metabolites, including plant metabolites, preferably cyclohexandiol epoxides, and of carbohydrate mimetics, preferably carba sugar, especially amino-carba sugar.
